(19) United States
(12) Patent Application Publication
(10) Pub. No.: US 2011/0166087 A1
(43) Pub. Date: Jul. 7, 2011

(54) PHARMACEUTICAL COMPOSITION FOR USE IN TREATING SEXUALLY TRANSMITTED INFECTIONS

(76) Inventors: Raúl García Salgado López, Colonia Anahuac (MX); Angélica Arzola Paniagua, Colonia Anahuac (MX); Juán Pablo Senosiain Peláez, Colonia Anahuac (MX); Gustavo Barranco Hernández, Colonia Anahuac (MX)

(21) Appl. No.: 13/062,182

(22) PCT Filed: Sep. 4, 2009
(86) PCT No.: PCT/IB2009/006738

§ 371 Date: Mar. 3, 2011

(30) Foreign Application Priority Data
Sep. 4, 2008 (MX) MX/A/2008/011321

(51) Int. Cl. A61K 31/7056; A61P 31/04; A61P 15/02

(52) U.S. Cl. 514/24

(57) ABSTRACT

The present invention relates to a solid oral pharmaceutical composition comprising fluconazole, tinidazole and clindamycin or a pharmaceutically acceptable salt thereof, wherein the content of pharmaceutically acceptable excipients is significantly lower than the content of the active principles, the use of said composition for oral administration with therapeutic activity for treating sexually transmitted infections.

### FIELD OF THE INVENTION

The present invention refers to a solid oral pharmaceutical composition comprising the combination of fluconazole, tinidazole and clindamycin and/or pharmaceutically acceptable salts thereof, wherein the content of the pharmaceutically acceptable excipients is significantly lower than the content of active agents; the use of said composition for oral administration with therapeutic activity for treating sexually transmitted infections.

### BACKGROUND OF THE INVENTION

Genital infections are one of the most common problems in gynecology, they are a group of infectious diseases caused by different types of microorganisms, whose common denominator is that they are mainly acquired through sexual intercourse and are called sexually transmitted infections (STIs).

These diseases do not have uniform effects, some can be serious, causing chronic pain, sterility, infertility, inflammatory pelvic disease; if they occur during pregnancy they may cause abortion, premature birth, ectopic pregnancy, among others. Therefore, if not prevented, diagnosed or treated early, they contribute to increased morbidity and mortality of women and men.

Prevalence studies about sexually transmitted infections (STIs) submitted by Villagrana-Zesati in “Sexually Transmitted Infectious Diseases, 2008” indicate that the two main infections are candidiasis and bacterial vaginosis, with incidences of 39% and 30% respectively, followed by infections whose etiologic agents are Ureaplasma urealyticum, Chlamydia trachomatis and Trichomonas vaginalis. It is worth to mention that mixed infection ranks third in frequency of infections, associating Candidiasis with Bacterial vaginosis or Candidiasis with Trichomoniasis.

Other reports from several national and foreign authors, indicate that within the fungal infections, Candida albicans is the most frequent agent, and within bacterial infections the most common is bacterial vaginosis followed by other organisms such as mycoplasma, Chlamydia and Trichomonas.

TABLE 1
Major diseases caused by sexually transmitted infections.

| | Causative Agent and | Treatment |
|---|---|---|
| Infection | Clinical Manifestation | (Regular Recommended Dose) |
| Vulvovaginal | The main causative agent is | Butaconazol 2%; vaginal cream for 3 |
| Candidiasis | Candida albicans; Others that | days. |
| | may occur: C. glabrata, | Clotrimazole 1%; vaginal cream or |
| | C. topicalis, C. parapsilosis. | tablet 7 to 14 days. |
| | Clinical manifestations | Clotrimazole vaginal tablet; two |
| | include: itching, burning, | tablets/day for 3 days. |
| | vulvar edema, fissures and | Isoconazole 1%; vaginal cream for 7 |
| | abrasions. | days |
| | The vaginal discharge is white, | Itraconazole 200 mg for 3 days. |
| | thick, lumpy, it is observed as | Miconazole 2%; vaginal cream for 7 |
| | attached to the cervix, to the | days. |
| | vaginal walls and to the vulva. | Nystatin 100000 I.U.; vaginal |
| | | tablet. One tablet for 14 days. |
| | | Tioconazole 6.5%; vaginal cream, |
| | | one dose. |
| | | Terconazole 0.4; vaginal cream 0.4% |
| | | for 7 days; 0.8% for 3 days. |
| | | Fluconazole 150 mg, tablet; a single |
| | | dose. |
| Bacterial | The main causative agent is | Antibiotics: |
| vaginosis | Gardnerella vaginalis and | Clindamycin cream 2%, a daily dose |
| | additionally anaerobic Gram- | for 5 days; |
| | negative bacilli. | Clindamycin 300 mg capsule; twice a |
| | Clinical manifestations: | day for 7 days; |
| | leukorrhea (whitish-gray, | Clindamycin ovules 100 mg; a daily |
| | homogeneous fluid, in moderate | dose for 3 days. |
| | or heavy amount). | Metronidazole; 2 capsules/day for 7 |
| | | days; gel application for 7 days. |
| Chlamydiasis | The main causative agent is | Azithromycin 1 g, single oral dose |
| | Gram positive bacteria | Clindamycin HCl, 450 mg, 4 doses/day |
| | Chlamydia in three species: C. | for 10 or 14 days |
| | Trachomatis, C. muridarum and | Erythromycin 500 mg, capsule, 4 |
| | C. suis | doses daily for 7 days. |
| | It is a strict intracellular | Ofloxacin, 300 mg capsule, twice a |
| | bacteria. | day for 7 days. |
| | Clinical manifestations: May be | Levofloxacin 500 mg, capsule, a |
| | asymptomatic. Sequels may | daily dose for 7 days. |
| | result in serious infections, | Doxycycline 100 mg, capsule, twice |
| | such as pelvic inflammatory | a day for 7 days. |
| | disease, ectopic pregnancy and | |
| | infertility. | |
| Trichomoniasis | The main causative agent is the | Metronidazole capsule, 2 g, single |
| | protozoan Trichomona vaginalis | dose; 500 mg twice a day for 7 days. |
| | | Tinidazole, 2 g capsule, a single |
| | | dose |

Chlamydia trachomatis is a strict intracellular bacteria, thus it was considered as a virus at the beginning of its identification. This condition obstructs its study because it cannot be cultivated on artificial media, therefore the use of cell cultures is the most suitable way for studying it, though it is not the most accessible one. Another technique consists in direct immunofluorescence. Currently, the Food and Drug Administration (FDA) approved the use of nucleic acid hybridization, because it proved to be the most sensitive.

The disease caused by Chlamydia is often asymptomatic, or the symptoms are so mild that many patients that have the disease do not realize it until they develop a more severe complication. It is estimated that 75% of women and 50% of men who are infected with Chlamydia do not notice any symptoms.

The symptoms in women, when present, are the following: abnormal leukorrhea, abnormal vaginal bleeding, pain or burning when urinating or pain in the lower abdomen, especially during sexual intercourse. Infection by Chlamydia in men can cause: testicular swelling or hypersensitivity, possibly symptoms of epididymitis which can cause infertility, urethritis, internal infection of the penis that can cause pain and urinating difficulty.

Mixed vulvovaginitis infection is the result of the association of two or more microorganisms, for example, the presence of candidiasis and bacterial vaginosis or candidiasis and trichomoniasis. In the case of mixed infection treatments, formulations have been developed, combining two active agents in short treatment schemes of 3 days up to the commonly used 7 days, as illustrated in Table 2.

TABLE 2
Combined Scheme for Treating Mixed Vulvovaginitis
Infection

| Active Agent | Dosage Form | Dose |
|---|---|---|
| Fluconazole- | Oral Tablet | Single dose |
| Tinidazole | 150 mg/2,000 mg | |
| Itraconazole- | Oral Capsules | Every 12 |
| Secnidazole | 33.3 mg/166.6 mg | hours for 3 |
| | | days |
| Tioconazole- | Vaginal tablet | Every 12 |
| Tinidazole | 100 mg/150 mg | hours for 3 |
| | | days |
| Clindamycin- | Ovules | 1 daily dose |
| Ketoconazole | 100 mg/400 mg | for |
| | | 7 days |

One of the agents used for treating vaginal infections is clindamycin, which is an antibiotic of the lincosamide family. It is a semisynthetic antibiotic that has a bacteriostatic effect and interferes with protein synthesis. Clindamycin has a half-life of 21 hours. Active clindamycin and its metabolites are excreted mainly in urine and some in bile.

Clindamycin is more effective against infections involving the following types of organisms:
Aerobic gram-positive cocci, including some staphylococci and streptococci (e.g. pneumococci).
Anaerobic gram-negative bacilli, including some members of the Bacteroides genera and Fusobacterium genera.
It attacks infections caused by Chlamydia.

Clindamycin may cause transitory adverse effects, although they can be moderately important. The most common effects are: nausea, vomiting, diarrhea, abdominal pain, flatulence, skin rash, itching.

Azithromycin is another antibiotic used against uncomplicated sexually transmitted infections due to Chlamydia Trachomatis.

In the state of the art, U.S. Pat. No. 7,094,431 refers to a pharmaceutical composition for treating skin repair, preferably by topical application, although oral administration is also mentioned. This formulation can be administered orally or not orally, and may contain: zinc oxide, fat soluble vitamins (A, D, E and K), an antibacterial agent, an antifungal agent and an effective amount of a calcium channel blocker such as nifedipine.

Unlike this document, the present invention is characterized by being an oral pharmaceutical composition comprising the combination of the active ingredients fluconazole, tinidazole and clindamycin, for the treatment of mixed vulvovaginal infection and sexually transmitted diseases in a daily dose.

US20050165077 patent document and its equivalent PA/a/0608279, refer to a composition containing fluconazole, tinidazole or secnidazole for treating vaginal infections of the type Gardnerella vaginalis, Actinomyces, Candida, Micrococcus, yeasts, Proteus, E. Coli, Trichomonas vaginalis, microorganisms present in vaginitis and bacterial vaginosis.

Unlike this document, the present invention addresses a broad spectrum of fungal and bacterial infections, and those caused by aerobic or anaerobic microorganisms such as Chlamydia, Gardnerella vaginalis, gram-negative and gram-positive bacilli and cocci, Peptostreptococcus, genital mycoplasmas and mobiluncus, with a high percentage of eradication and effectiveness in a very short scheme of treatment.

The novel combination of fluconazole, tinidazole and clindamycin has not been reported to date. In the state of the art there are several studies that include combinations of drugs that do not fully address, and without a high degree of effectiveness, the conditions of sexually transmitted infections or mixed vulvovaginal infections.
Malhotra (2003) worked on combinations of ciprofloxacin, and tinidazole applied for seven days and fluconazole-azithromycin-secnidazole in the form of a kit, and the combination of doxycycline and metronidazole for several days.
Ariella Baylson (2004) conducted a study about the treatment with tinidazole for recurrent infections caused by bacterial vaginosis.
Livengood (2007) studied two tinidazole treatment schemes for bacterial vaginosis.
Say, P (2005) conducted studies about the difficulty of treating vaginitis.

Unlike these studies for the treatment of vaginal infections, the present invention refers to an oral pharmaceutical composition of fluconazole, tinidazole and clindamycin, useful in the treatment of sexually transmitted infections. This composition is applied in a single day, has a wide spectrum and results in a decrease of infectious relapse.
Workowski, K. A. et. al (2006, 55 RR11) in their document “Sexually Transmitted Disease Treatment Guidelines. Morbidity and Mortality Weekly Report 2006; 55, RR11”, presents a guide for the treatment of sexually transmitted infections (STIs). In this guide there are individual and combined treatments that eradicate several mixed vulvovaginal-cervical conditions. However, the time period for the combined treatment is more than one day and additionally the guide does not mention the use of the combination of fluconazole, tinidazole and clindamycin.

The novel combination of fluconazole, tinidazole and clindamycin has not been reported to date. The present pharmaceutical invention exhibits significant advantages over existing formulations for STIs, such as:
A broad-spectrum effect, because the combination allows the physician to safely and reliably attack (in pharmaceutical terms) the mixed vulvovaginosis infections without having to wait for the laboratory test results.
Decreasing the possibility of failure due to discontinuation of treatment, because it involves a simplified scheme of a single day treatment.
The one day treatment is more easily accepted by the couple as compared with other treatments that last more than one day, such as the treatment with clindamycin, which according to the prior art, is administered at least twice a day for 7 days.
The combination is completely absorbed in the gastrodigestive tract, which allows achieving appropriate plasma concentrations without any competition between the active ingredients. This effect occurs with or without food ingestion, this results in no limitations at the moment of administering the product.
The fluconazole-tinidazole-clindamycin combination does not interact in the process of elimination, or in the average life of each one of the active ingredients. It was found that this allows for an adequate contribution of therapeutic effects in the combination, without compromising patient health.
The pharmaceutical composition has at least one active agent in an amount less than the regular total dose, as is the case of using a lower dose of clindamycin in a single day treatment, which leads to fewer adverse effects.
The combination is stable and meets pharmaceutical specifications such as: appearance, content uniformity, dissolution, valuation; this is achieved despite the fact that the composition contains a higher content of active agents than the content of excipients.
In the present invention, the pharmaceutical composition is physicochemically stable and meets pharmaceutical specifications. In a preferred embodiment, the content of excipients is not greater than 40% by weight of the dosage unit.

### JUSTIFICATION OF THE INVENTION

The need to attack a broad spectrum of microorganisms by combining fluconazole, tinidazole and clindamycin is socially useful because it can be used in villages that lack of the technical or economic conditions for performing clinical tests, and that only count on medical diagnosis for treatment.

Surprisingly, in the present invention, the composition with the association of fluconazole, tinidazole and clindamycin is therapeutically effective, although clindamycin is at a significantly lower total dose compared with the regular doses, and although it is administered in a one-day treatment. This fact improves the scheme of attention of these diseases with respect to the known treatments. The therapeutic scope for the spectrum of microorganisms includes Gardnerella vaginalis and Chlamydia, which results in a significant advantage over longer-term treatments because it ensures treatment compliance by the patient.

The regular oral dose of clindamycin for the treatment of vaginal infections is 300 mg to 450 mg per day for 7 days or more. However, in the present invention, clindamycin is administered in a one-day treatment, at total doses of 300 mg to 2400 mg, a preferred total dose might be 1200 mg to 1800 mg.

A technical challenge overcome by this invention is the association of fluconazole, tinidazole and clindamycin or suitable pharmaceutically salts thereof in a single unit dose and a lower content of excipients.

Additionally, this composition offers a highly therapeutic effectiveness that allows a rapid reduction of the symptoms, it is well tolerated and more acceptable by the patient.

Another challenge overcome by the present invention is to offer the combination of fluconazole, tinidazole and clindamycin in a dosage unit of an acceptable size so that it can be swallowed by the patient.

For the aforementioned reasons, the present invention provides a pharmaceutical composition for preparing a very useful drug for treating sexually transmitted infections using the combined effect of the active agents of the composition.

The therapeutic effectiveness provided by the combination of fluconazole, tinidazole and clindamycin against infections such as bacterial vaginosis, candidiasis, chlamydiasis, tricomoniaisis, mycoplasma, and/or mixed vulvovaginitis, makes this invention an effective alternative for treating these conditions with a therapeutic scheme of a single day treatment.

The present invention offers an oral formulation, preferably tablets, where is important to highlight that the active agents fluconazole, tinidazole and clindamycin do not have good compression properties nor fluidity. One would expect that during the development of the formulation, it may be necessary to add excipients in order to compensate the lack of compression and fluidity.

Evidently, if less excipients are used, it is more difficult to achieve fluidity and compressibility. Additionally, a lower percentage of excipients in the formulation reduces the possibility of modifying physical properties such as appearance, consistency, dissolution rate, content uniformity.

In the present invention it is possible to obtain a physicochemically stable formulation that meets fluid compressibility specifications, appearance, content uniformity, dissolution rate, and which contains a smaller amount of excipients, for example, below 40%.

### DETAILED DESCRIPTION OF THE INVENTION

The formulation and administration of drug combinations is not easy, because when administering two or more active ingredients in a single dosage form, interactions between the active agents, adverse reactions, side effects, incompatibility (physic, chemical and physicochemical), as well as technological problems resulting from the physicochemical interaction of the active agents themselves and with the excipients, may occur.

In accordance with the above description, it can be seen that the broad spectrum, the treatment shortness and the effectiveness of the active ingredients combination, as well as the security of having a pharmaceutical form with all the criteria of quality, are factors of great importance for giving benefits to the patients with an appropriate treatment for these diseases.

Formulations

The formulation and manufacturing process of the pharmaceutical composition of fluconazole, tinidazole and clindamycin or pharmaceutically acceptable salts thereof, additionally, pharmaceutically acceptable carriers or excipients, are described below:

Generally, for the tablet formulation, the active ingredients fluconazole, tinidazole and clindamycin do not exhibit suitable fluidity and compressibility properties.

In the present invention is possible to obtain a physicochemically stable formulation that meets the specifications of formulation stability.

To manufacture the present invention, different excipients were tested, such as: compressibility carriers, microcrystalline cellulose, lactose, starch; diluent binders, lactose, dextrose, sucrose, mannitol, polividone; antistatics, sodium lauryl sulfate, silicon dioxide, talc; disintegrants, croscarmellose sodium, crospovidone, sodium starch glycolate; lubricants, magnesium stearate, magnesium phosphate, stearic acid, glyceryl stearate, polyethylene glycol, sodium stearyl fumarate, talc; plasticizers, propylene glycol, polyethylene glycol, among other glycol derivatives; ligand polymer binders, hypromellose, polyvinyl pirrolidone, hydroxypropyl cellulose; coating polymers, methacrylate copolymer derivatives (Opadry), polyvynilpirrolidone, hydroxypropylmethylcellulose; coating and finishing polymer, cellulose and methacrylate derivatives (Opaglos), polyvinylpirrolidone, polyvinyl alcohol, polyethylene glycol, hydroxypropylmethylcellulose. Within all the excipients, equivalent excipients and/or mixtures thereof were tested.

### FORMULATION EXAMPLE

Table 3 presents a general formulation of the triple combination. Tables 4 and 5 illustrate examples of formulations comprising fluconazole, tinidazole and clindamycin, where the amounts by weight of active ingredients, carriers and/or excipients may be used within the mentioned ranges.

TABLE 3
General oral tablet formulation

| | RANGE OF USE |
|---|---|
| | WEIGHT PERCENT |
| COMPONENTS | PER UNIT DOSE |
| Tinidazole | 40 to 55% |
| Fluconazole | 1 to 4% |
| Clindamycin | 30 to 65% |
| Compressibility carriers | 2 to 12% |
| Diluent binder | 0.1 to 2.6% |
| Disintegrant | 0.05 to 4% |
| Antistatic | 0.1 to 1% |
| Binding ligand polymer | 1.5 to 4% |
| Lubricant | 0.2 to 0.4% |
| Coating and finishing | 0.0 to 2% |
| polymer | |
| Isopropyl alcohol | — |
| Purified water | qs |

TABLE 4
Formulation 1, oral tablets with clindamycin.

| | Weight | | |
|---|---|---|---|
| | Percent | Content | |
| | per Unit | (mg) per | |
| Components | Dose | Unit Dose | |
| Tinidazole | 50 | 500 | mg |
| Fluconazole | 3.7 | 37.5 | mg |
| Clindamycin (as clindamycin | 32.5 | 312.5 | mg |
| hydrochloride) | | | |
| Microcrystalline cellulose PH 102 | 7.5 | 85 | mg |
| Sodium starch glycolate | 0.6 | 6.5 | mg |
| Crospovidone | 1.6 | 17 | mg |
| Sodium lauryl sulfate | 0.6 | 7 | mg |
| Polyvinylpyrrolidone | 1.9 | 20 | mg |
| Magnesium stearate | 0.3 | 3.2 | mg |
| Methacrylate derivatives (Opadry | 1 | 10.5 | mg |

| | | |
|---|---|---|
| White) | | |
| Isopropyl alcohol | — | — |
| Purified water | qs | qs |

TABLE 5
Formulation 2, oral tablets with clindamycin.

| | Weight | Content | |
|---|---|---|---|
| | Percentage/ | Mg/Unit | |
| Components | Unit Dose | Dose | |
| Tinidazole | 40 | 500 | mg |
| fluconazole | 3 | 37.5 | mg |
| Clindamycin (as clindamycin | 36 | 450 | mg |
| hydrochloride) | | | |
| Microcrystalline cellulose pH 102 | 10.4 | 130 | mg |
| Sodium starch glycolate | 2 | 25 | mg |
| Crospovidone | 3.8 | 48 | mg |
| Sodium lauryl sulfate | 0.8 | 10 | mg |
| Polyvinylpyrrolidone | 3.6 | 45 | mg |
| Magnesium stearate | 0.3 | 3.7 | mg |

| | | |
|---|---|---|
| Isopropyl Alcohol | — | — |
| Purified water | qs | qs |

### Manufacturing Process

The manufacturing process for the combination of active agents in a single solid phase is a novel method for the incorporation of fluconazole, a wet granulation in which tinidazole is incorporated and clindamycin for further compression.
1) Formulation components are weighed.
2) Binder solution is prepared by dissolving in water: isopropyl alcohol, fluconazole, polyvinyl pyrrolidone, sodium lauryl sulfate.
3) Sieved apart: Tinidazole, third active principle, microcrystalline cellulose, sodium starch glycolate and crospovidone.
4) The materials from step 3 are mixed in a “V” mixer.
5) The mixture obtained in step 4 is granulated in a fluidized-bed equipment, with the binder solution of step 2.
6) The obtained granulate is sieved.
7) Crospovidone and sodium lauryl sulfate are added to the granulate.
8) The mixture from step 7 is granulated.
9) Magnesium stearate is added to the mixture from step 8 and then mixed.
10) The mixture from step 9 is compressed to obtain a tablet.
11) The coating is prepared separately, by adding water to the methacrylate derivative.
12) The coating is applied on the tablets obtained in step 11.
13) The obtained product is conditioned.

The process can be used with other solid oral compositions which can be, without limitation, granules and capsules. In fact, the maximum values of the use ranges shown in Table 3 are considered suitable for granulates. The process for such granulation is the same as the one previously mentioned for preparing tablets, but without the tabletting steps.

The tablets were submitted to a stability evaluation, the results are shown in Table 6.

TABLE 6
Physicochemical Evaluation of the Formulations

| | | | | Weight |
|---|---|---|---|---|
| | | | | Percent |
| | Evaluation | | Dose | per |
| Formulation | Time | Appearance | Uniformity | Unit Dose |
| 1 | Starting | Oblong tablet | Complies | 99% |
| | point | with no stains or | | |
| | | spots | | |
| 2 | Three | Oblong tablet | Complies | 98% |
| | months | with no stains or | | |
| | | spots | | |

As previously described, it is observed that the tablets comply with stability requirements.

The combination of fluconazole-tinidazole-clindamycin is effective for the treatment of the most common germs found in the clinical practice of the reproductive tract, such as Candida albicans, Gardnerella vaginalis, Chlamydia, Trichomonas vaginalis, among others.

The composition with fluconazole-tinidazole-clindamycin combination, has fewer side effects due to the lower amount of clindamycin used (1250 mg), compared to the regular recommended dose for treatment.

The invention has been sufficiently described so that a person of ordinary skill in the subject matter can reproduce and obtain the results mentioned in this description. However, any person skilled in the art of this invention may be able to make modifications not described in this application. However, if the implementation of these modifications in a given composition requires the claimed matter in the following claims, such compositions should be included within the scope of the present invention.

1.-11. (canceled)

12. A pharmaceutical combination comprising tinidazole, fluconazole and clindamycin or pharmaceutically acceptable salts thereof.

13. A pharmaceutical combination comprising tinidazole, fluconazole and clindamycin or pharmaceutically acceptable salts thereof, for the treatment of sexually transmitted infections and mixed vulvovaginal infections.

14. The pharmaceutical combination of claim 13; wherein the combination is suitable for a one-day treatment.

15. A pharmaceutical composition for oral administration comprising tinidazole, fluconazole and clindamycin or pharmaceutically acceptable salts thereof, for the treatment of sexually transmitted infections or mixed vulvovaginal infections.

16. The pharmaceutical composition of claim 15, wherein the composition is suitable for a one-day treatment.

17. The pharmaceutical composition of claim 15 wherein the composition is suitable for administration in solid form.

18. The pharmaceutical composition of claim 17, wherein the composition is suitable for administration in tablets.

19. The pharmaceutical composition of claim 15, wherein clindamycin or its pharmaceutically acceptable salts are present between 30% to 65% by weight per unit dose.

20. The pharmaceutical composition of claim 15, further comprising fluconazole, tinidazole and between 300 to 2400 mg of clindamycin or pharmaceutically acceptable salts thereof.

21. The pharmaceutical composition of claim 15, further comprising pharmaceutically acceptable excipients or at least one of the following excipients: microcrystalline cellulose, sodium starch glycolate, crospovidone, sodium lauryl sulfate, polyvinylpyrrolidone, magnesium stearate, methacrylate derivatives (Opadry White) and isopropyl alcohol.

22. A method of treating sexually transmitted infections and/or mixed vulvovaginal infections comprising the step of administering the pharmaceutical composition of claim 15 to a patient in need thereof.

23. The method according to claim 22, wherein the pharmaceutical composition is administered as a one-day treatment.

24. The method according to claim 22, wherein the pharmaceutical composition is administered in solid form.

25. A process for the manufacture of the pharmaceutical composition of claim 15, comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.

26. The process of claim 25, further comprising the step of compressing the granulate.

27. A process for the manufacture of the pharmaceutical composition of claim 16, comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.

28. A process for the manufacture of the pharmaceutical composition of claim 17, comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.

29. A process for the manufacture of the pharmaceutical composition of claim 18; comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.

30. A process for the manufacture of the pharmaceutical composition of claim 19, comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.

31. A process for the manufacture of the pharmaceutical composition of claim 20, comprising the steps of:
preparing a binder solution by mixing fluconazole, binding ligand polymer, microcrystalline cellulose, disintegrant, antistatic and lubricant;
mixing separately, tinidazole and clindamycin with excipients such as microcrystalline cellulose, starch sodium glycolate, disintegrant, ligand polymer binder and antistatic; and
adding the components to a mixer and obtaining a granulate.
